(19) United States
(12) Patent Application Publication
(10) Pub. No.: US 2026/0331284 A1
(43) Pub. Date: Oct. 22, 2026

(54) Compliance-Gated Multimodal AI Diagnostic System

(71) Applicant: George William Bickerstaff, III, Greenwich, CT (US)

(72) Inventor: George William Bickerstaff, III, Greenwich, CT (US)

(21) Appl. No.: 19/378,428

(22) Filed: Nov. 4, 2025

(51) Int. Cl. G16H 50/20; G06N 3/098; H04L 9/32

(52) U.S. Cl. CPC G16H 50/20; G06N 3/098; H04L 9/3239

(57) ABSTRACT

A computer-implemented diagnostic system integrates multimodal data—including imaging, biochemical, genomic, and behavioral sources—to detect and monitor neurological disorders. The system enforces ledger-recorded quorum approval prior to deploying model updates, ensuring regulatory compliance and auditability. Adaptive multimodal fusion with uncertainty handling enables robust and interpretable AI diagnostics under a compliance-gated governance framework.

### FIELD OF THE INVENTION

The invention relates to artificial-intelligence-based diagnostic systems and, more particularly, to computer-implemented methods for adaptive detection, assessment, and monitoring of neurodegenerative and central-nervous-system (CNS) disorders using multimodal data sources under a verifiable compliance-gated governance framework.

### BACKGROUND OF THE INVENTION

Conventional diagnostic systems relying on single modalities, such as MRI, PET, or blood assays, fail to capture the multi-dimensional nature of early-stage disease.

Existing federated learning systems—such as FedAvg and Owkin—enable decentralized training but lack ledger-governed quorum gating. Model updates are aggregated automatically without mandatory approval, limiting auditability and regulatory transparency.

Such architectures risk model drift, bias propagation, and untraceable update cycles.

There remains a need for a transparent, audit-ready system that fuses multimodal inputs under adaptive uncertainty control and enforces deployment through cryptographically recorded approval—creating a compliance-gated, regulator-verifiable loop.

### SUMMARY OF THE INVENTION

The invention discloses a Compliance-Gated Multimodal AI Diagnostic System integrating adaptive multimodal fusion with uncertainty handling and immutable ledger-based governance.

Unlike prior federated systems, the invention enforces ledger-recorded quorum approval before deploying any model update.

The architecture comprises:
(a) a Data Acquisition Module (110) for encrypted multimodal ingestion;
(b) a Diagnostic Engine (120) executing adaptive multimodal fusion with uncertainty handling; and
(c) a Compliance Layer (130) maintaining a ledger-enforced quorum gate that must approve model updates prior to deployment.

The quorum threshold, validation criteria, and adaptive parameters evolve as scientific, clinical, and regulatory standards change, maintaining future alignment without redesign.

This structure provides a verifiable, compliance-gated continual learning cycle that prevents unverified model drift and enables regulator-auditable decision transparency.

### LIST OF FIGURES AND SUBFIGURES

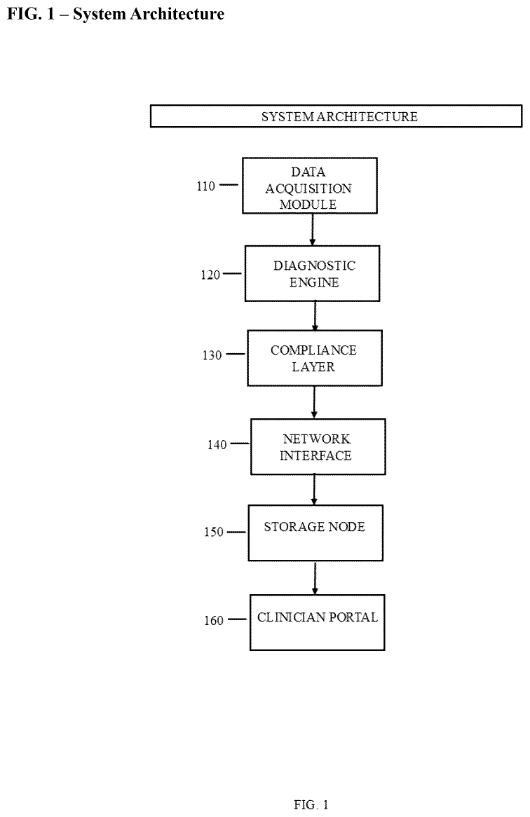

FIG. 1—System Architecture Overview
110 Data Acquisition Module
120 Diagnostic Engine
130 Compliance Layer
140 Network Interface
150 Storage Node

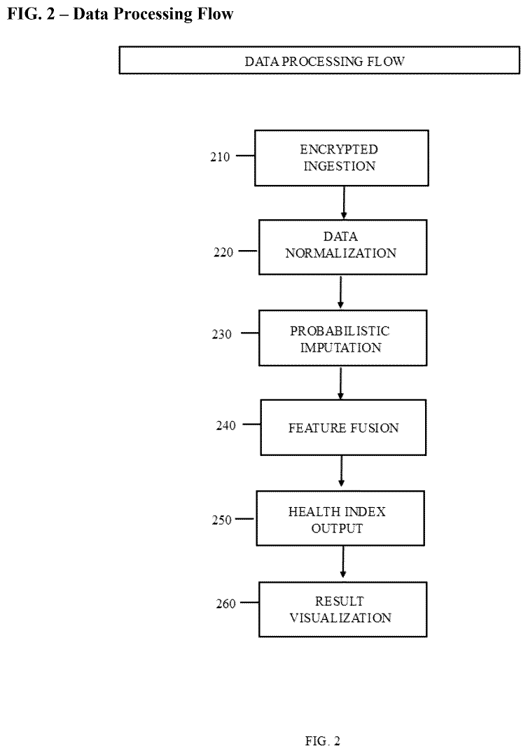

FIG. 2—Data Processing Flow
210 Encrypted Ingestion
220 Preprocessing Layer
230 Feature Extraction
240 Feature Fusion
250 Health Index Output
260 Result Display

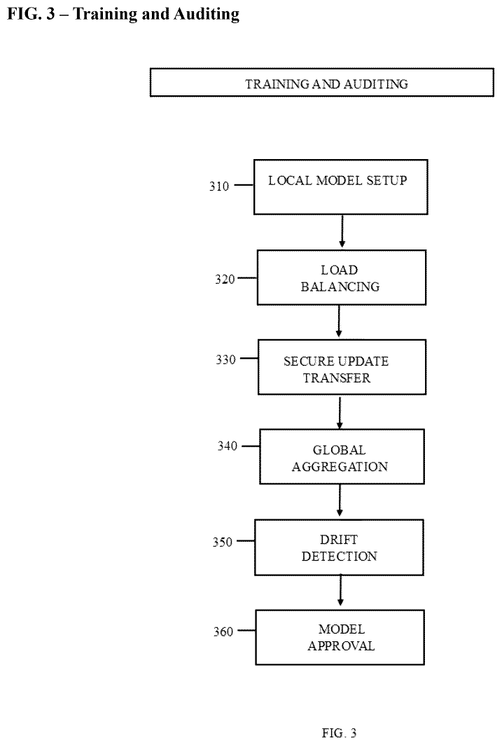

FIG. 3—Training and Audit Cycle
310 Local Model Setup
320 Local Training Phase
330 Secure Update Transfer
340 Performance Validation
350 Drift Detection
360 Model Approval Process

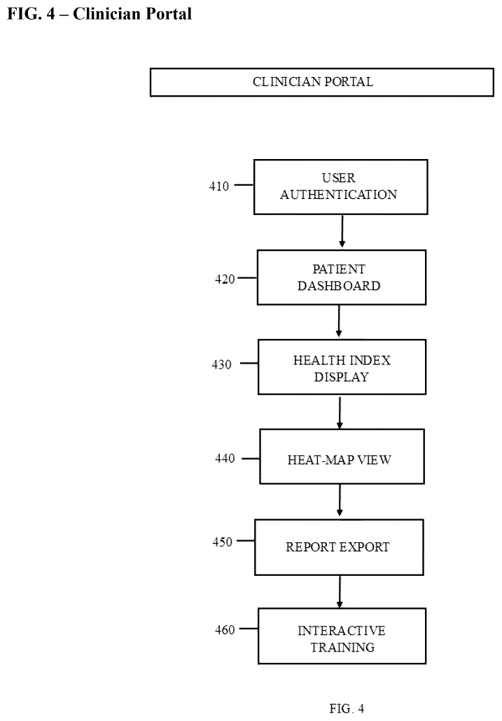

FIG. 4—Clinician Portal and Visualization
410 User Authentication
420 Patient Dashboard
430 Visualization Engine
440 Model Insight Panel
450 Ledger Trace Viewer
460 Training Insight Panel

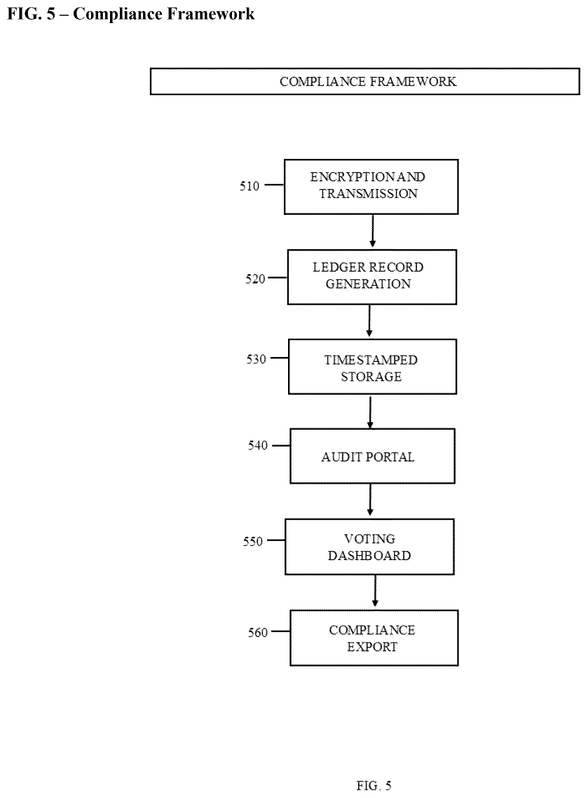

FIG. 5—Compliance and Governance Framework
520 Ledger Record Generation
530 Timestamped Storage
540 Audit Portal
550 Voting Dashboard
560 Compliance Export

### DEFINITIONS

Adaptive Parameter—A system variable automatically or manually adjusted based on new data, clinical results, or regulation.
Ledger—A cryptographically secured, immutable sequence of records (e.g., SHA-256 hash-linked) capturing all model training, validation, and approval events.
Quorum—A configurable approval threshold defining the number or proportion of nodes or reviewers required to authorize deployment.
Simulated-Federated Learning—A local emulation of federated environments for validation prior to production deployment without live data sharing.
NeuroHealth Index—An interpretable score derived from multimodal fusion representing a patient's neurological health trajectory.

### DETAILED DESCRIPTION OF THE INVENTION

### System Architecture (FIG. 1)

The system operates as a distributed modular architecture. The Data Acquisition Module (110) securely ingests multimodal data including imaging, biochemical, genomic, and behavioral signals.

The Diagnostic Engine (120) fuses these modalities using adaptive uncertainty weighting and generates a clinical interpretation.

The Compliance Layer (130) integrates with the Network Interface (140) and Storage Node (150) to record all actions in a cryptographic ledger. No model update propagates without ledger-approved quorum consensus.

### Data Processing Flow (FIG. 2)

Encrypted Ingestion (210) ensures end-to-end security. The Preprocessing Layer (220) and Feature Extraction (230) normalize and prepare multimodal inputs.

Feature Fusion (240) combines weighted representations according to uncertainty measures. Health Index Output (250) generates an interpretable NeuroHealth Index, and Result Display (260) visualizes results with provenance traceability.

Each inference event produces a ledger-stamped record linking data origin, model version, and output context.

### Training and Audit Cycle (FIG. 3)

Local Model Setup (310) initializes models within each participating node, followed by Local Training Phase (320) and Secure Update Transfer (330).

Performance Validation (340) evaluates metrics locally before aggregation. Drift Detection (350) identifies deviations beyond adaptive thresholds.

Model Approval Process (360) triggers when drift or update events occur, requiring ledger-recorded quorum approval before integration. The quorum threshold is policy-configurable and stored on the ledger.

This transforms ordinary training loops into verified, regulator-auditable continual learning cycles.

### Clinician Portal and Visualization (FIG. 4)

User Authentication (410) ensures authorized access. The Patient Dashboard (420) displays longitudinal results.

The Visualization Engine (430) renders interpretive graphics such as risk timelines, confidence zones, and trend heat maps.

The Ledger Trace Viewer (450) allows clinicians to access the underlying ledger record of each model decision. The Training Insight Panel (460) presents performance summaries and model provenance for full transparency.

### Compliance and Governance Framework (FIG. 5)

Ledger Record Generation (520) captures each event—training, validation, or update—in immutable hash-linked form.

Timestamped Storage (530) ensures chronological order and tamper protection.

Audit Portal (540) and Voting Dashboard (550) execute the quorum approval workflow.

Compliance Export (560) generates regulator-readable documentation, including version histories, signatures, and approval timestamps.

AES-256 encryption and SHA-256 hashing provide confidentiality and verifiable integrity for all governance transactions.

### Example Embodiment

In one embodiment, three hospital networks train localized models for early Alzheimer's prediction. Each model passes Drift Detection (350) and must receive two-thirds quorum approval under Model Approval Process (360).

Validation occurs in Simulated-Federated mode before deployment, ensuring interoperability and data protection.

Parameters, thresholds, and governance criteria evolve automatically as new clinical, scientific, or regulatory insights emerge, while maintaining immutable traceability through the ledger.

### Advantages of the Invention

(a) Governance Novelty: The compliance-gated quorum approval system introduces regulator-verifiable AI oversight absent in prior federated systems.
(b) Adaptive Intelligence: Employs multimodal fusion with uncertainty handling for robustness under incomplete data.
(c) Auditability: Immutable ledger records each event from ingestion to deployment.
(d) Regulatory Readiness: Aligns with evolving audit and change-control standards.
(e) Scalability and Privacy: Supports modular expansion and cryptographic security across global healthcare networks.

1. A computer-implemented diagnostic system comprising:
(a) a data-acquisition module for encrypted multimodal data ingestion;
(b) a diagnostic engine executing adaptive multimodal fusion with uncertainty handling to generate interpretable clinical outputs; and
(c) a compliance layer maintaining an immutable ledger, wherein deployment of any model update is conditioned on ledger-recorded quorum approval.

2. The system of claim 1, wherein the ledger-based quorum mechanism distinguishes the architecture from federated learning systems that aggregate updates without verified compliance gating.

3. A method for compliance-gated multimodal diagnostics comprising:
(a) collecting and encrypting multimodal data across distributed nodes;
(b) performing adaptive multimodal fusion with uncertainty handling;
(c) detecting model drift;
(d) halting model aggregation pending ledger-recorded quorum approval; and
(e) displaying a NeuroHealth Index to authorized users.

4. The system of claim 1, wherein the ledger stores hash-linked entries containing timestamps, drift metrics, and node identifiers.

5. The method of claim 3, wherein quorum threshold is configurable and stored as a ledger policy parameter.

6. The system of claim 1, wherein the compliance layer employs AES-256 encryption and SHA-256 hashing for data integrity.

7. The system of claim 1, wherein the diagnostic engine supports additional data modalities without architectural redesign.

8. The method of claim 3, wherein the system operates in a simulated-federated learning environment for validation before deployment.
